(19) United States
(12) Patent Application Publication
(10) Pub. No.: US 2008/0288077 A1
(43) Pub. Date: Nov. 20, 2008

(54) Prosthetic Disc Assembly Having Natural Biomechanical Movement

(75) Inventors: Michael L. Reo, Redwood City, CA (US); Elisa Bass, San Francisco, CA (US); Darin C. Gittings, Sunnyvale, CA (US); Nicholas C. Koske, San Jose, CA (US); Roxanne L. Richman, San Jose, CA (US); Dean Carson, Mountain View, CA (US)

Correspondence Address: Wheelock Chan LLP, P.O. Box 61168, Palo Alto, CA, 94306, US

(73) Assignee: Spinal Kinetics, Inc., Sunnyvale, CA (US)

(21) Appl. No.: 11/966,955

(22) Filed: Dec. 28, 2007

Related U.S. Application Data
(60) Provisional application No. 60/877,558, filed on Dec. 28, 2006.

(51) Int. Cl. A61F 2/44

(52) U.S. Cl. 623/17.16; 623/17.11; 623/17.15

(57) ABSTRACT

Described here is a surgical device. Specifically, the device is a prosthetic spinal implant that replaces a natural disc in the spine. The device has biomechanical attributes substantially similar to a natural disc.

### FIELD

We describe a surgical device. Specifically, the device is a prosthetic spinal implant that completely replaces a natural disc in the spine. The device has biomechanical attributes substantially similar to a natural disc, whether cervical or lumbar.

### BACKGROUND

The natural intervertebral disc is an anatomically and functionally complex joint. The functional joint is made up of three component structures: (1) the nucleus pulposus; (2) the annulus fibrosus; and (3) the bony vertebral end plates. The biological composition and anatomical arrangements of these component structures are major factors in the biomechanical functioning of the disc. Additionally, and to further complicate the understanding of a disc's functioning, the movement of an individual disc in the spine in response to outside or to muscular forces is affected by the functioning and responsive movement of adjacent discs in the spinal structure.

The various responsive motions of a natural disc, caused by exterior forces or those forces coming from the musculature, measured as functions of rotation or displacement between the two vertebrae adjacent a specific disc, are exceedingly complex. Measurements of each of the forces (or moments) required to flex and to restore a natural disc in the front-to-back direction (flexion-extension), in the side-to-side direction (lateral bending or in the saggital plane), and in torsional or twisting rotation, exhibit a non-linear relationship between the force and movement. In addition to the lack of mere linearity in the various relationships between applied force and resultant translational or rotational movement in the vertebrae adjacent a specific disc, each of the relationships includes a region near the midpoint in the movements, typically called the “neutral zone” in which little or no force is needed to move those adjacent vertebrae from their natural resting points. See, for instance, the discussion of the “neutral zone” in Panjabi, “The Stabilizing System of the Spine, Part II, Neutral Zone and Instability Hypothesis,” Journal of Spinal Disorders, 1992, vol. 5, no. 4, pp. 390-397 and in U.S. Pat. No. 7,029,475, to Panjabi.

The paths of movement or rotation of each of the vertebrae adjacent a disc during these various flexures and rotations are very complex. As a vertebral bone is moved, that vertebral bone movement is not a mere circular movement. The ligaments of the disc, the facet joints associated with the disc, the disc's nucleus pulposa, and surrounding tissues all contribute to the complexity of the vertebral motion. The geometrical collection of these axes of rotation (as the observed vertebra is moved) forms a very complicated locus. This geometrical collection of the Instantaneous Axes of Rotation (IAR) is not a single point nor is it a single line except during an instantaneous movement. For instance, the IAR of various cervical vertebrae move significant distances during flexion and extension of the spine. See, Mameren H. van, Sanches H., Beursgens J., Drukker, J., “Cervical Spine Motion in the Sagittal Plane II: Position of Segmental Averaged Instantaneous Centers of Rotation-A Cineradiographic Study”, Spine 1992, Vol. 17, No. 5, pp. 467-474. This quantified motion varies widely amongst the various spinal joints in an individual spine and amongst individuals. The motion further depends on age, time-of-day, and the general health and condition of the intervertebral discs, facet joints, and other components of the spine. A moving IAR means that a vertebral bone both rotates or translates while moving with respect to a lower (or adjacent) vertebral member. Natural spinal motions place severe requirements on the design of a prosthetic disc; simple rotational joints are not able meet those requirements.

In addition, there is an amount of motion coupling between axial and lateral bending. To some extent, the structure and placement of the facet joints also influence the motions of adjacent interconnecting vertebrae and also constrain flexion-extension, side-to-side, and axial motions. The orientation of the facet joints varies in the spine and induces wide variations in motion parameters and constraints.

Finally, the natural disc itself exhibits significant, elastic, compressibility. The height or thickness of the disc may become smaller during the active time of the day; similarly, the disc size regenerates during resting time.

In the event that a natural spinal disc is to be replaced, a replacement prosthetic disc having biomechanical properties (rotation and compressibility) substantially similar to the native disc provides the best opportunity for overall success of the disc replacement.

If a natural disc is displaced or damaged due to trauma or disease, the nucleus pulposus may be herniated and protrude into the vertebral canal or into intervertebral foramen. Such deformation is commonly known as a herniated or “slipped” disc. The herniation may be of such an extent that it presses on a spinal nerve as it exits the vertebral canal through the partially obstructed foramen. Such a condition may cause pain or even paralysis in the area of the nerve's influence.

Prior treatments or procedures for slipped discs included a procedure known as “spinal fusion.” This procedure has been extensively used in the past and is still currently used to alleviate the condition. The procedure involves surgically removing the involved disc and fusing together the two adjacent vertebrae. In this procedure, a spacer or spacers are inserted in the place originally occupied by the disc and the spacers are secured by screws and plates or rods attached to the vertebrae. Although “spinal fusion” is an excellent treatment, in the short-term, for traumatic and degenerative spinal disorders, various studies have shown that in the longer term, immobilization of a specific disc site leads to degenerative changes at the adjacent discs. Spinal fusion also often leads to excessive forces on facet joints adjacent to the fusion. Those adjacent spinal discs incur increased motion and stress due to the increased stiffness of the fused segment. In the long term, this change in the mechanics and of the motion of the spine causes the degeneration. Obviously, this treatment does not restore normal disc function.

Artificial intervertebral disc replacement devices have been proposed as alternatives to spinal fusion. None of the various types of those artificial intervertebral discs are believed to provide the normal kinematics and load-sharing properties of the natural intervertebral disc.

Artificial discs of the ball and socket type are usually made up of metal plates, one metal plate to be attached to the upper vertebra and the other to be attached to the lower vertebra, and including a polymeric, often polyethylene, core working as a ball. The metal plates have concave areas to house the polyethylene core. The ball and socket type disc allows free rotation between those adjacent vertebrae, that is, between the two vertebrae between which the prosthetic disc is installed. This disc design does not have the capability of absorbing a load imposed on the spine when the spine undergoes a bending motion. Additionally, artificial discs of this type are typically not compressible along the spinal axis. Such a lack of load-bearing capability often causes degeneration in adjacent discs, since those adjacent discs must shoulder significant portions of the extra loads passed on from the ball and socket artificial disc.

Additionally, ball-and-socket designs such as shown in Salib et al., U.S. Pat. No. 5,258,031; Marnay, U.S. Pat. No. 5,314,477; Boyd et al., U.S. Pat. No. 5,425,773; Yuan et al., U.S. Pat. No. 5,676,701; and Larsen et al., U.S. Pat. No. 5,782,832 limit motion to rotation only about the socket when the two plates are in contact. Some studies e.g., Bogduk N. and Mercer S., “Biomechanics of the cervical spine. I: Normal kinematics”, Clinical Biomechanics, Elsevier, 15 (2000) 633-648; and Mameren H. van, Sanches H., Beursgens J., Drukker, J., “Cervical Spine Motion in the Sagittal Plane II: Position of Segmental Averaged Instantaneous Centers of Rotation-A Cineradiographic Study”, Spine 1992, Vol. 17, No. 5, pp. 467-474, note that this restricted motion does not correspond to the natural motion of the vertebrae, even for side-to-side motion.

In certain of the elastic rubber type artificial discs, an elastomeric polymer is embedded between and affixed to metal end plates and those metal plates are, in turn, affixed to adjacent upper and the lower vertebrae. The elastomeric polymer is bonded or affixed to a rough and porous interface surface the metal end plates. This type of disc can absorb a shock in the vertical direction and has a load-bearing capability. However, the interfaces between the elastomeric polymer and the opposing metal plates found in this structure often generate polymeric debris after long term usage. Furthermore, the elastomer may shear or rupture after long usage due to insufficient shear-fatigue strength at the metal end plates.

The rocker arm devices (Cauthen, U.S. Pat. Nos. 6,019,792 and 6,179,874) appear to have motion and stability limitations as do the sliding disc cores found in the Bryan et al. patents (U.S. Pat. Nos. 5,674,296; 5,865,846; 6,001,130; and 6,156,067) and the CHARITE disc, as described by Buettner-Jantz K., Hochschuler S. H., McAfee P. C. (Eds), The Artificial Disc, ISBN 3-540-41779-6 Springer-Verlag, Berlin Heidelberg New York, 2003; and U.S. Pat. Nos. 4,759,766 and 5,401,269 to Buettner-Jantz et al. In addition, the sliding disc core devices of the Bryan et al. and CHARITE devices do not permit natural motion of the joint for any fixed shape of the core.

In particular, the CHARITE discs' sliding core, in some cases, generates precipitous constraining forces by restricting closure of the posterior intervertebral gap. Furthermore, the core does not mechanically link the upper and lower plates of the prosthesis and does not maintain the intervertebral gap throughout the range of motion. In general, such accentuated relative motion between the two vertebral plates in that prosthetic disc eventually contribute to disc instability.

Again, certain prosthetic discs absorb only minimal static loading. For example, load bearing and shock absorption in the CHARITE design and others (e.g. Bryan et al., U.S. Pat. No. 5,865,846) rely on the mechanical properties of the resilient, ultra-high-molecular-weight polyethylene core to provide both strength and static and dynamic loading to the joint. The rigidity of the sliding core appears to offer little energy absorption and appears not to provide sufficient flexibility in maintaining an appropriate intervertebral gap during joint motion. Such a design most likely generates excessive reaction forces on the spine during flexion, forces that potentially produce extra stress on facet joints and affect mobility.

The limits of rotational movement in the spine during flexion-extension, side-to-side, and angular movements have been widely studied. See, Mow V. C. and Hayes W. C., Basic Orthopaedic Biomechanics, Lippincott-Raven Pub., N.Y., 2nd Ed., 1997. However, the text, while describing angular limits, does not discuss the underlying complex relational motion between two adjacent vertebrae during that movement. The article, Mameren H. van, Sanches H., Beursgens J., Drukker, J., “Cervical Spine Motion in the Sagittal Plane II: Position of Segmental Averaged Instantaneous Centers of Rotation-A Cineradiographic Study”, Spine 1992, Vol. 17, No. 5, pp. 467-474 shows the complexity of these movements in the cervical spine, particularly in flexion and extension.

Later kinematic models of spinal movements using a mechanical preload along the curving axis of the spine have provided a superior method for understanding and quantifying the forces and rotational movements of individual spinal discs. See, Patwardhan AG, et al. “Load-carrying capacity of the human cervical spine in compression is increased under a follower load.” Spine 2000; 25:1548-54.

None of the cited patents or literature is believed to show an artificial disc having biomechanical attributes similar to those of a natural disc.

### SUMMARY

Described here is a prosthetic intervertebral replacement disc or disc assembly having at least three components: upper and lower (or “first” and “second”) end components that are directly or indirectly affixable to adjacent vertebrae in the spine and a specific compressible core member assembly (or core structure) that cooperates with the two end components in such a way that the resultant assembly includes at least the listed biomechanical attributes of a natural disc and substantially mimics the operation of that natural disc.

The described disc is designed to purposefully mimic the physiologic movement of a natural disc. A healthy natural disc's range of motion (ROM) involves complex coupled motions. The described prosthetic disc will fit into the local biomechanical profile provided by adjacent vertebral bodies, ligaments, and facet joints. Our prosthetic disc assumes the kinematic characteristics of the replaced natural disc.

In particular, as one or the other of the end components is subjected to a force or moment from muscle or exterior sources, the rotation of that end component follows a rotational or translational path that is determined by the compressibility of, and by the tension of, portions of the core component acting upon those end components.

In addition to an end component's cooperating to pass forces across the compressible core member to the other end component and, as appropriate for the level of force, to cause motion in that other end component, the upper and lower end components are configured such that they operate to respond to motions of, or to cause motions in, the vertebral bones to which they are affixed. Typically, the end components will act as a fixed portion of the bones to which they are attached, having no relational movement between bone and end plate.

The core structure, containing one or more compression elements and one or more stress elements or one or more integrated elements, is cooperatively linked to first and second end components in such a way that our prosthetic disc exhibits nonlinear mechanical responses of a specific form to specific forces (or moments) applied to end components of the prosthetic disc. In particular, the non-linear mechanical responses include a region (“neutral zone”) in a central region of the disc's movement.

Specifically, the responsive movement of our prosthetic disc is not defined by the contact of a pair of hard or bearing surfaces contacting each other.

The core structure, in one variation, may comprise one or more stress components that transmit stress between or relative to the first and second end components. The core structure either comprises or is the sole structure providing tension between the end components upon movement of those end components. In this variation, the stress component may be configured so that it provides substantially none of the overall compressibility to the prosthetic disc. In this variation, the core structure may further comprise one or more compression elements that provide substantially all of the compressibility to the prosthetic disc, as viewed between the end components. In this variation, the compression elements may be configured such that they provide none of or substantially none of the tension between the first and second end components.

The stress components may comprise fibers, wires, membranes, fabrics (woven or nonwoven) and secured to the first and second and components in such a way that least a portion of the stress component provides tension between the first and second and components.

In a further version of the disc, the stress members or stress components may provide some amount of compressibility to the assembled disc.

The stress components may variously be independent of, in contact with, or integrated into one or more compression elements.

For placement in the spine, the end components may be directly or indirectly affixed or connected to the adjacent vertebrae. As assembled, the end components, alone or with other ancillary components, move in conjunction with those adjacent vertebrae as if they were those vertebrae. Such ancillary components may include, for instance, devices that are attached to (or are attachable to) the end components and have functions such as fixation of the end components directly to the vertebrae, securement of core components or subcomponents to the end components, placement of the subassembly comprising the end components and the core structure at a desired position in the spine between the vertebrae, and the like.

Although we may utilize many different devices or materials to affix the end components to the vertebrae, e.g., adhesives, screws, pins, expanding rivets, etc., the choice should be one that minimizes both the amount of vertebral bone removed and the potential for harm to the bone during implantation or later use. The fixation components may comprise barbed keels.

### BRIEF DESCRIPTION OF DRAWINGS

FIG. 1 shows a portion of a spine having two vertebrae, a natural disc, facet joints, and a set of axes useful in understanding the motion concepts described elsewhere.

FIG. 2 schematically shows a variation of our prosthetic disc.

FIG. 3 schematically depicts another variation of our prosthetic spinal disc.

FIG. 4A shows, in schematic fashion, a cross-section of our disc at rest. FIG. 4 B. shows a cross-section of our prosthetic disc under load.

FIG. 5 shows a graph of typical moment versus range of motion relationship for our prosthetic disc.

FIGS. 6A and 6B show, respectively, a perspective view and a cross-section view of a variation of the stress member.

FIG. 7 shows a complementary set of end plates for the FIGS. 6A and 6B stress member.

FIGS. 8A and 8B show, respectively, a perspective view and a side cross-section view of another variation of our stress member.

FIG. 9 shows the complementary set of end plates for the stress member shown in FIGS. 8A and 8B.

FIGS. 10A and 10B show, respectively, a perspective view and a side cross-section of still another variation of our stress member.

FIG. 11 shows a pair of complementary end plates for use with the stress member shown in FIGS. 10A and 10B.

FIGS. 12A and 12B show, respectively, a perspective view and a side cross-sectional view of another variation of our stress member.

FIG. 13 shows a pair of end plates suitable for use with the stress member shown in FIGS. 12A and 12B.

FIGS. 14 and 15 show cross-sectional views of compression members suitable for our prosthetic disc.

FIGS. 16, 17, and 18 show devices or subcomponents useful in affixing our prosthetic disc to the spine.

FIG. 19A is an exploded view, in perspective, of one desirable variation of our prosthetic disc omitting however, the stress members.

FIG. 19B is an exploded view, in perspective, of another desirable variation of our prosthetic disc.

FIG. 20 shows, in perspective, a fiber-wound version of the core structure as may be used, for instance, in the devices shown in FIGS. 19A and 19B.

FIG. 21 shows a test setup for measuring biomechanical values of a prosthetic disc while inserted in a spine.

FIG. 22 provides a graph showing typical results from the testing of our device.

FIG. 23 provides a number of hysteresis load-displacement curves in a cadaver spine. The curves compare those values, in flexion-extension, for the C5-C6 location of an intact spine and of one of our prosthetic discs implanted at that location in that same spine.

FIG. 24 shows bar graphs of the testing of our prosthetic disc in a cadaver spine.

### DESCRIPTION

Described here is a prosthetic spinal disc or spinal disc assembly that is intended to closely approximate the biomechanical movement of a healthy disc. In general, this prosthetic assembly may be placed in a slot or opening formed by the surgical removal of anatural vertebral disc and of a minimum amount of vertebral bone from each of the two vertebrae adjacent the disc. When the prosthetic disc is introduced anteriorly, some amount of surrounding ligament surrounding the disc site, i.e., laterally and posteriorly, is often left intact. It is into this slot that our prosthetic spinal disc is desirably placed. Our prosthetic spinal disc is configured to mimic the biomechanical movement that would be shown by a healthy disc removed from that slot, whether the disc is located in the cervical or lumbar spine.

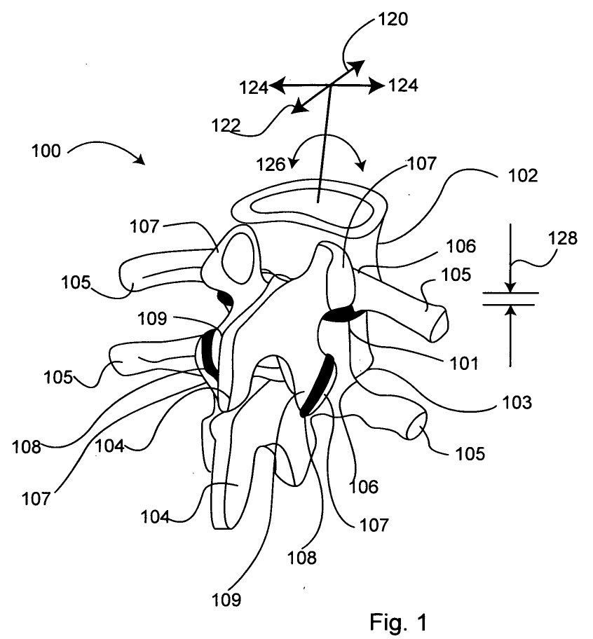

FIG. 1 shows a portion of a human spine (100) having an upper vertebral body (102) and a lower vertebral body (103) separated by a disc (101). Each of the two vertebral bodies (102, 103) also includes spinous processes (104), transverse processes (105), and pedicles (106). Articular processes (107, 109) supporting the facet joints (108) are also shown. Each vertebral body (102, 103) includes two sets of articular processes, the superior articular processes (109), and the inferior articular processes (107). The facet joints (108) cooperate with the disc (101) to allow and to limit movements of the spine including flexion (bending forward), extension (bending backward), side-to-side, and twisting motions. The stability of the spine is enhanced due to the interlocking nature of the adjacent vertebrae (102, 103).

Imposed on the spinal unit (100) shown in FIG. 1 is a set of reference coordinates that may be used to quantify the motion of a vertebral body (102), for instance, with respect to the other vertebral body (103) and within spinal unit (100). As mentioned above, the movement of a vertebra such as vertebral body (102) is quite complex. If the lower vertebral body (103) is considered (for purposes of explanation and of providing a basis for a relative system of coordinates) to be motionless, and the upper vertebral body (102) is moved in flexion (120), extension (122), from side to side (124), and twisted (126) about its axis, the motions would not be simple circular rotations or linear movements. The effects of the positioning (or geometry) of the facet joints (108) with respect to the disc (101), their respective compressibilities, and other related anatomical features all mandate a responsive motion of the upper vertebral body (102) that is quite complex.

Additionally, disc (101) has a measure of compressibility shown in FIG. 1 at (128). The value for a healthy natural cervical disc is 737 N/mm+/−885 N/mm.

The responsive motions in flexion (120), extension (122), and from side-to-side (124) are generally rotational in nature. However, as mentioned elsewhere, this rotation includes an instantaneous center or axis of rotation. The viscous and elastic nature of the disc and varying effect of the facet joints on the vertebral body movement causes this complexity. Our prosthetic disc mimics the movement of a natural disc in response to external forces or moments. In our disc, the specific responsive movements are due to the choice of materials, their compositions, certain of their physical parameters (compressibility, geometry, etc.), situated in the prosthetic disc core assembly and, in some cases, the matter in which they are attached to the assembly.

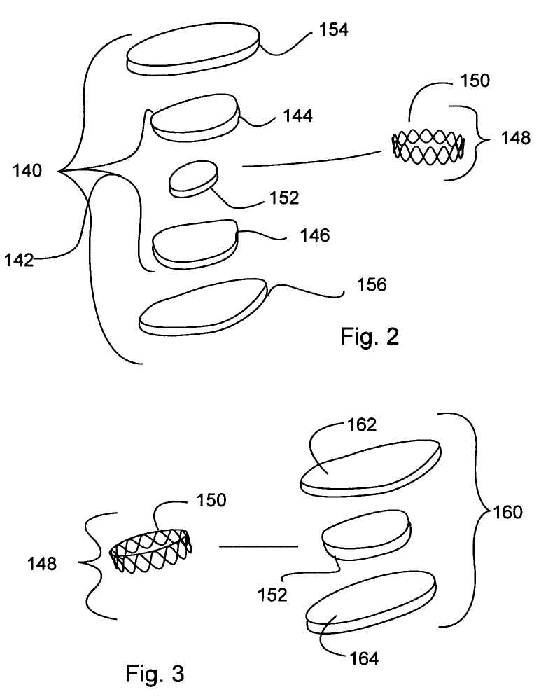

FIGS. 2 and 3 show schematic representations of our prosthetic disc, specifically, FIG. 2 provides a version of our disc using end components that are indirectly attachable to vertebral bodies via other members tailored specifically to fasten the combined end components to the vertebral bone. FIG. 3 provides a schematic representation of a variation in which the end components are attachable directly to the vertebral bone.

FIG. 2 provides an exploded view of a variation (140) having a prosthetic disc assembly (142) that is, in turn, made up of an upper end component or end plate (144), a lower end component or end plate (146), a core assembly (148) made up of a stress component (150) and a compression component (152), an upper attachment member (154), and a lower attachment member (156).

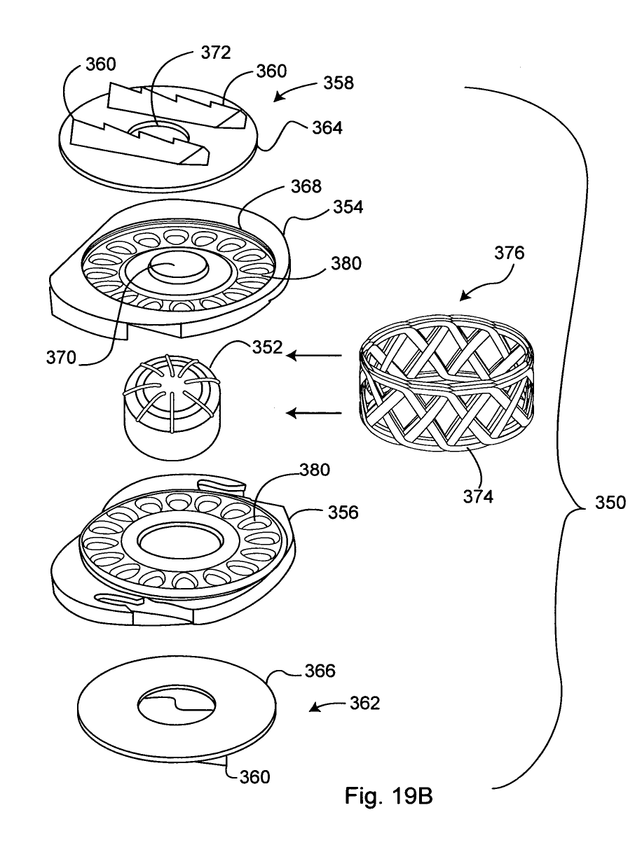

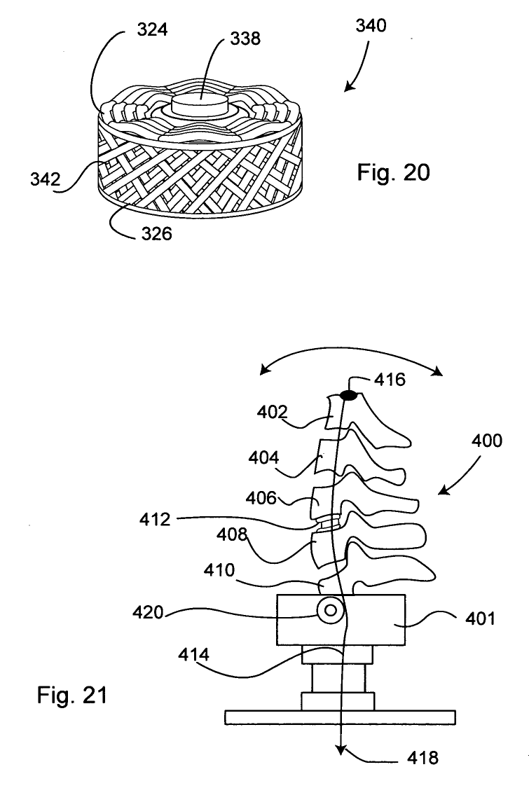

As will be discussed below in greater detail, the stress component (150) is attached to the upper and lower end components (144, 146) in such a way that as the two end components (144, 146) are moved, rotated, or twisted with respect to each other, a specific relationship between the force or moment applied and a resulting movement is established. Taking part in this relationship is compression member (152). Compression member (152) for a cervical human implant, typically is a compressible, rubbery, or elastomeric component having a compressibility of about 737 N/mm+/−885 N/mm. For a human lumbar implant, the compressibility value is about 1200 N/mm+/−600N/mm. Desirably, the compression member has limited physical compression under physiologic loads, over time. That is to say: although the compression member may or will slowly compress during time, e.g., a day, under the load of normal use, there is a limit to the compression or compression member height or thickness. Suitable compositions for the compression member (or members) will be discussed below. For a compression member having a nominal thickness of approximately 2.00 mm to 3.50 mm—as may be used in a cervical disc implant—a compression of 0.0 mm to about 1.0 mm (leaving at least about 1.00 mm of compression member thickness) is observed with the variation shown in FIGS. 19A and 20. For a compression member having a nominal thickness of approximately 8-12 mm—as may be used in a lumbar disc implant—a similar compression is observed.

In the variation shown in FIG. 2, the stress member (150) is a filamentary component affixed in some fashion to the upper attachment member (154) and the lower attachment member (156). Typically, although not necessarily, compression component (152) is not affixed to end member (144) nor to end member (146).

In the FIG. 2 variation, end members (144, 146) will be functionally quite stiff and thereby not participate in the resulting moment-movement relationship for the prosthetic disc assembly (140), other than to provide a solid or predictable foundation for the attachment of stress component (150) and compression sites for compression member (152). Suitable materials for end members (144, 146) include such biologically acceptable materials as titanium, titanium alloys (e.g., with aluminum or tungsten or the like), stainless steels, certain ceramics, and certain polymers (engineering plastics, filled polymers, or reinforced polymers). One particularly suitable material is a widely known titanium alloy (Titanium—6% of Aluminum—4% of Vanadium (Ti 6A1-4V)). This titanium alloy has been a material of choice for medical implants, particularly orthopedic implants, for decades. This alloy is generally considered chemically inert, compatible with human tissue, and resistant to corrosion by human body fluids.

We have also had good experience with coating at least the bone contact areas of our device with a titanium plasma spray to increase bone-contact surface area. The titanium spray material comprises commercially pure titanium. Other materials may be suitable for increasing the surface area of the bone contact areas.

Attachment members (154, 156) may comprise materials similar to those used for end members (144, 146). One species of attachment members (154, 156) is shown and discussed below with regard to FIG. 19A and others. The manner of attaching end members (144, 146) respectively to attachment members (154, 156) may be left to a designer of a particular configuration using these teachings. Often though, the manner in which the end members are attached to the attachment members is mechanical or physical in nature, e.g., slots, screws, pins, etc., allowing a firm bond between the two. In turn, the manner of affixing attachment members (154, 156) to vertebral bone is a matter for the designer of a specific device. We have had good results with the barbed keels depicted below in FIG. 16 and further in FIGS. 19A and 19B. Various alternative devices and subcomponents for affixing the attachment members and hence the prosthetic disc to the adjacent vertebral bodies and are shown below.

FIG. 3 shows a variation of the prosthetic disc (160) in which the functions of the end components and the attachment components as shown in FIG. 2 are combined into a single member. A specific variation of this conceptual design is discussed below with regard to FIG. 19B.

The variation (160) shown in FIG. 3 also includes a core assembly (148) that is similar in makeup and operation to that shown in FIG. 2. The stress member (150), in this variation, is attached to upper combination plate (162) and to lower combination plate (164). Combination plate members (162, 164) further include some means, devices, or materials allowing attachment to the adjacent vertebral bodies. Common to this variation and to the others described herein is the described relationship between an applied force or moment to one of the end members or combination members and the resulting movement to that end member or combination member in our assembled prosthetic disc assembly.

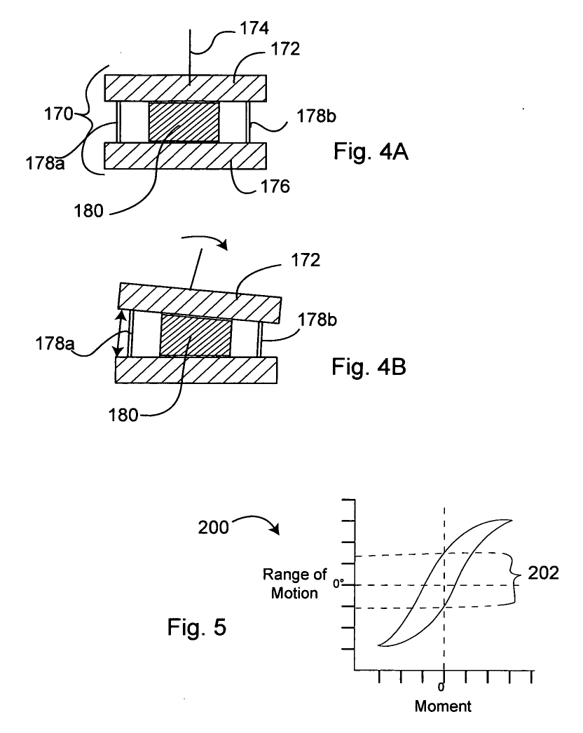

FIGS. 4A and 4B show, in a schematic fashion, the ways in which application of the force to an end member results in motion of that member and the concomitant effects upon the stress member and compression member found in the core assembly. FIG. 5, in turn, provides a graphic representation of the movement-force relationship and in particular shows both the nonlinearity of the relationship and the presence of a “neutral zone” generally centered in that motion. The form of the relationship, as depicted in FIG. 5, is the same in each of flexion-extension, side-to-side motion, and rotational or twisting motion.

FIG. 4A schematically shows one of our prosthetic discs (170) having upper end plate (172) with an arbitrary pole (174) depicting a defined axis of that upper end plate (172), and further having a lower end plate (176), schematic stress members or components of the stress members (178) and compression element (180). This schematically depicted disc may be considered to be assembled from the components shown in the exploded view found in FIG. 3. Obviously, a useful prosthetic disc will typically have more than the two stress members (or stress member components) as shown in FIG. 4A, but the remainder of the stress members have been removed from this depiction for the purpose of explanation. In this schematic assembly, the stress members (178) are fixedly attached to the significantly stiffer upper end member (172) and a lower end member (176). The compressible core member (180) provides all of the compressibility in this schematic disc variation.

As a force is applied to upper end member (172), from left to right, some portion of the compressible member (180) is compressed, the left stress member (178a) is stretched and the other stress member (178b) in the right of the depiction is relaxed or the amount of stress on (178b) is at least reduced.

In the variation shown in FIG. 20, the number of filaments lengths serving as components of the stress element passing from end plate to end plate, number at around a hundred. The distributed nature of the force transmission from end plate to end plate may be appreciated. The forces applied between the two end plates, when viewed on an individual fiber level, should be understood to vary from at (or near) none for some fibers to the high stress value likely situated at the site of maximum movement.

In our device, when the appropriate materials are chosen, the generalized relationship shown in FIG. 5 is appropriate whether the force is applied from side-to-side, in flexion-extension, in rotation, or in combinations of those directions.

FIG. 5 shows, in graph (200), the generalized relationship of the rotatory motions exhibited by our device when subjected to various forces or moments. The hysteresis exhibited in graph (200) also shows the self-restoring (or self-centering) feature of the prosthetic disc. The hysteresis provides for a “zone” (202) in which the prosthetic disc allows a range of motion of a few degrees without the application of substantial force. In a natural disc, the extent of the neutral zone in the three noted motions, i.e., flexion-extension, side-to-side, and axial twisting, changes with such variables as: the location of the disc in the spine, age, disease state, and to a lesser extent, time of day and level of health. Cervical discs have an extensive neutral zone (e.g., +4° to −2.5° in flexion-extension) and ultimate range of motion (ROM) limits (e.g., 10° to 16° in flexion-extension; 8° to 10° in lateral movement; and 8° to 10° in axial rotation) in each of the noted motions.

Lumbar discs generally have a less extensive neutral zone (e.g., +/−2°) in flexion-extension) and ultimate range of motion (ROM) limits (e.g., +/−10° to 12° in flexion-extension; +/−10° in lateral movement; and +/−6° in axial rotation) in each of the noted motions.

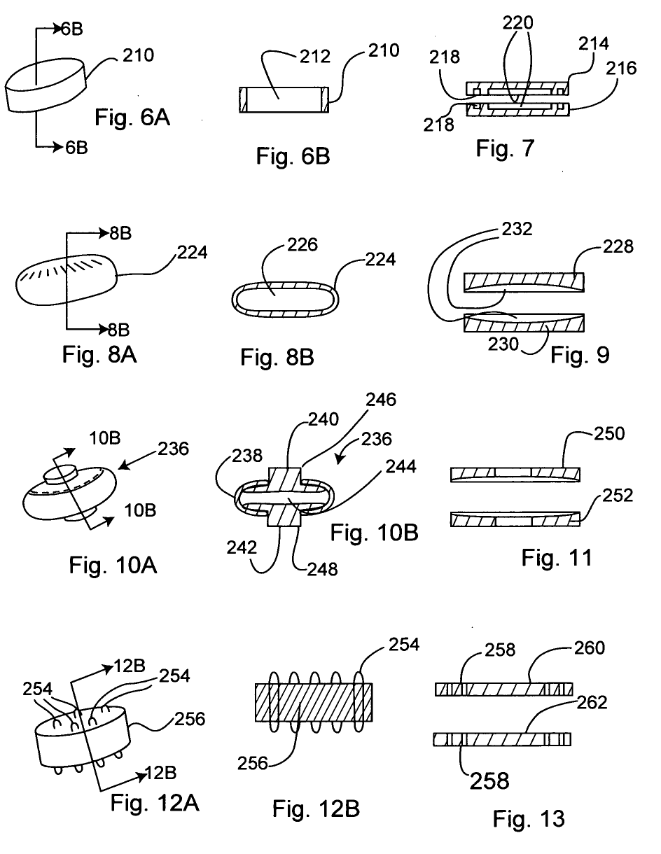

FIGS. 6A-13 show several variations of the subcomponent found in the core assembly that we have designated as the core stress member and the end components that are complementary to the specific depicted core stress member. We have discussed the use of fibers as components of the stress member above and will do so again below. In any variation of our device, the stress member is affixed to the end members in such a way that the stress member conducts force both axially across the core element from one end member to the other as a flexion-extension or side-to-side moment is impressed upon an end member and also conducts force from end member to end member as one of them is twisted. This is a major difference from most existing prosthetic disc devices.

FIGS. 6A shows, in perspective, a stress member (210) that comprises a fabric that may be woven or un-woven. FIG. 6B shows a cross section of the stress member (210). Stress member (210) includes a hollow region (212) into which the compressible member(s) may be situated.

FIG. 7 shows an upper end member (214) and a lower end member (216), each including matching grooves into which the edges of stress member (210) may be inserted and affixed there by such methods as interfering-fit mechanical rings or by appropriate adhesives. Shallow pockets (220) are shown in each of the upper and lower end members (214, 216).

FIG. 8A shows, in perspective, a stress member (224) that is a closed bag or sack. A cross section of stress member (224) is shown in FIG. 8B. Stress member (224) includes an open region or volume (226) into which one or more compression members may be placed upon assembly.

FIG. 9 shows an upper end member (228) and a lower end member (230), each having small depressions (232) that are complementary in general shape to the upper and lower surfaces of the compression member (224) shown in FIG. 8A. The stress member (224) may also be affixed to the upper end member (228) and to the lower and member (230) by adhesives. In this way, forces applied to one or the other of end members (228, 230) may be transferred through the adhesive layer, into the stress member (224), through any core compression member that may be situated there, and finally into the other end member. The variously distributed forces on one end member causes some amount of motion in the other end member by that transmission of force across the core member assembly.

FIG. 10A shows a perspective view of another variation of a stress member, in this case, including a portion (240, 242) of end members within the stress member's interior volume. Stress member (236) is shown in perspective in FIG. 10A. FIG. 10B provides a cross section of stress member (236) showing a fabric bag or sack (238), which fabric may be woven or unwoven. An upper subcomponent (240) and a lower subcomponent (242) are shown to be enclosed within the volume situated within stress component member (238). That open volume is also used to contain one or more compressive core members (244). The upper and lower subcomponents (240, 242) are shown to have curving surfaces in the region where they contact the stress component bag member (230). And although this curving form may be desirable in some variations, it is not necessary. The fabric bag or sack (238) may be adhesively attached to the upper subcomponent (240) and to the lower subcomponent (242). Similarly, the exterior of the bag (238) may be adhesively attached to the end plates (250, 252) shown in FIG. 11. The extensions (246, 248), respectively, on upper subcomponent (240) and on lower subcomponent (242) may each be welded to their respective end plates (250, 252) shown in FIG. 11. Either or both of these fixation methods (adhesives, welding) may be used to assure that the stress member moves with the end plates. Other procedures may be used to attach the upper and lower subcomponents (240, 242) to their respective end plates (250, 252), e.g., the extensions (246, 248) may be threaded to match threads in the end plates, the extensions (246, 248) may be radially expanded to form an interference fit in the openings in end plates (250, 252).

FIG. 12A shows a perspective view of another variation of the stress member. In this variation, the stress member is collectively distributed through a number of independent loops (254) that are integrated into the compression member (256). The loops (254) extend through the compression member (256) and are attached to openings (258) in upper end plate (260) and lower end plate (262) shown in FIG. 13.

In some variations of our prosthetic disc, the stress member may, due to its bulk or inherent stiffness, provide some measure of compressibility to the prosthetic disc assembly in addition to that provided by the compression member alone. A substantial portion of the compressibility of the prosthetic disc assembly will always be provided by the compression member (or members).

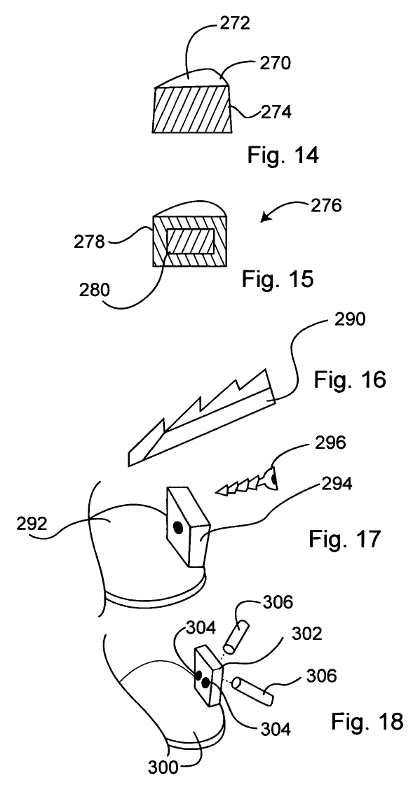

FIGS. 14 and 15 show perspective cross-sectional views of compression members suitable for use in our prosthetic spinal disc.

FIG. 14 shows such a compression member (270). The compression member (270) typically has a generally cylindrical shape, as will be shown in discussion of another variation, a barrel. In this variation, the depicted compression member is a solid and has a consistent composition throughout. This compression member (270) is shown to be a single component. In other variations, the compression member (270) may be two or more subcomponents. As noted elsewhere, the compression member in a cervical implant may be an elastomeric material having a compressibility of 737 N/mm+/−885 N/mm. Grooves or furrows may be incorporated into the upper side (272) or outer side (274) if needed for gas or ethylene oxide sterilization. In particular, the compressible core member may be thermoplastic elastomer (TPE) such as a polycarbonate-urethane TPE having, e.g., a Shore value of 50 D to 60 D, e.g. 55 D, such as the commercially available TPE, Bionate. Shore hardness is often used to specify flexibility or flexural modulus for elastomers.

For a comparable lumbar implant disc, the compression member may comprise the same compositional material in the disclosed designs, but the compressibility may instead be 1200N/mm+/−600N/mm.

We have had success with compression members comprising TPE that are compression molded at a moderate temperature beginning with an extruded plug. For instance, with the polycarbonate-urethane TPE mentioned above, a selected amount of the polymer is introduced into a closed mold upon which a substantial pressure may be applied, while heat is applied. The TPE amount is selected to produce a compression member having a specific height. The pressure is applied for 8-15 hours at a temperature of 70°-90° C., typically about 12 hours at 80° C. For a cervical disc, a typical nominal compression member height may be 2.00 to 3.5 mm. For a lumbar disc, a typical nominal compression member height in this variation may be 7.00 to 8.00 mm, more typically about 7.5-7.7 mm. The lumbar disc compression member width in this variation may be 18.0-19.0 mm., more typically 18.4-18.6 mm.

FIG. 15 shows a composite core member (276) having an outer layer (278), typically of material such as a TPE, and an inner portion (280) comprising, e.g., a suitable hydrogel, viscous fluid, or other fluid.

FIGS. 16, 17, and 18 show various subcomponents or devices useful in affixing our prosthetic spinal disc to a spine.

FIG. 16 shows a barbed keel (290) that may be integrated into the end plates shown, for instance, in FIGS. 7, 9, 11, and 13 and is specifically shown in FIG. 19. These barbed keels (290) may be sized to fit in keel tracks or slots that are cut or chiseled into cortical bone of the spine. The keels need not be very tall to allow immediate and acute fixation by means of a press fit into the slots or keel tracks. For instance, we have found that keel heights of approximately 1.8 mm are quite effective for immediate and for long-term fixation.

FIG. 17 shows an end plate (292) having a securing tab (294) integrated therein. A bone screw (296) is used to secure the implant into the spinal bone.

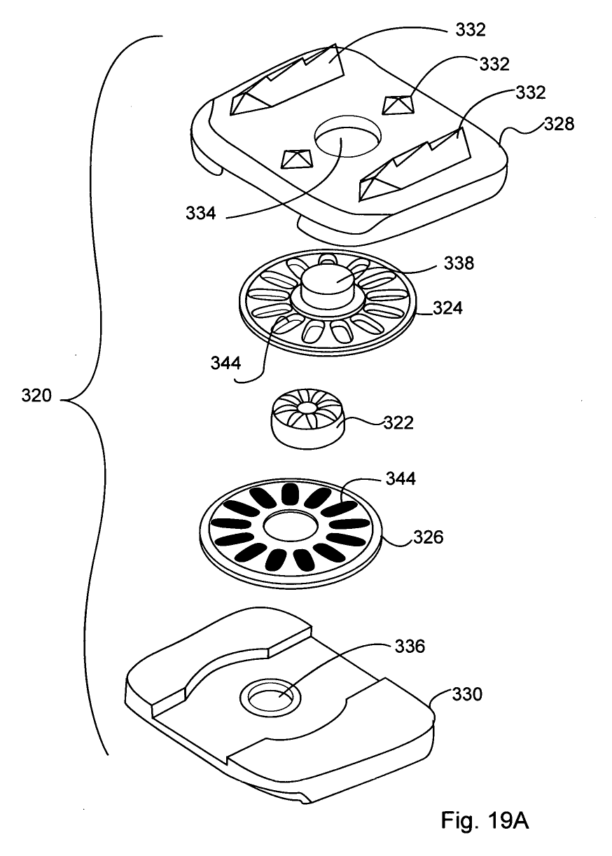

FIG. 18 shows still another variation in which end plate (300) is equipped with an integral tab (302) having canted openings (304) allowing the insertion of pins (306) to form a “V” behind the tab (302) in previously prepared holes in the cortical bone of the vertebral body adjacent end plate (300) and thereby fix the prosthetic disc to the spine.

FIG. 19A provides an exploded view of a desirable variation of our prosthetic disc (320). The stress member is not shown in this figure but will be discussed with regard to FIG. 20. Shown in FIG. 19A is a particular disc assembly of the class shown in FIG. 2 and discussed above. Included are a compression member (322) and upper inner end plate (324), a lower inner end plate (326), an upper outer end plate (328), and a lower outer end plate (330). The upper outer end plate (328) includes the integrated barbed keels (332) discussed above with regard to FIG. 16. Lower outer end plate (330) also includes barbed keels but are located on the non-visible side of the end plate (330). Upper outer end plate (328) and lower outer end plate (330) each include an opening, respectively (334) and (336) into which the cylindrical protrusions (338) on upper inner end plate (324) fit. The upper outer end plate (328) and the upper inner end plate (324) maybe welded along the periphery of opening (334). Similarly, a protrusion (338) extending from lower inner end plate (326) may be welded into the opening (336) of lower outer end plate (330). Such a welding step takes place after the assembly of a subassembly made up of the upper inner end plate (324), the compression member (322), and the lower inner end plate (326), using, e.g., the woven fibers discussed below. Such a subassembly (340) may be seen in FIG. 20.

FIG. 19B provides an exploded view of another variation (350) of our prosthetic disc. Shown in FIG. 19B is a specific variation of the class of discs shown in FIG. 3 and discussed above. Included is a compression member (352), upper end plate (354), and lower end plate ((356). An upper end cap (358) including fixation members, i.e., barbed keels, and a lower end cap (362) also including barbed keel fixation members (360) are also shown. In this variation, the respective end caps (358, 362) fit into and may be affixed to the upper and lower end plates (354, 356). In the variation shown, the outer edges (364, 366) of the end caps (358, 362) fit into cooperating openings, e.g., (368) in upper end plate (354), and welded at their junction. Similarly, the protuberance (370) on end plate (354) fits into the opening (372) of upper end cap (358) and their junction is also welded. A similar pair of welds is also provided for lower end plate (356) and lower end cap (362). As is the case with the variation shown in FIG. 19A, the welding steps take place after the fibers (374) of stress member (376) have been woven through the slots or openings (380) in the end plates (354, 356) and placement of the compression member or core (352) between the end plates (354, 356).

As is the case with subassembly (340) discussed elsewhere with regard to FIG. 20, the stress member (374) comprises one or more filaments woven through the openings or slots or openings in upper and lower end plates (354, 356) surrounding the compressible core member (352). Together, in this variation, they form the core assembly.

Subassembly (340) in FIG. 20 is also referred to as a core structure and comprises the upper inner end plate (324), the inner lower end plate (326) discussed above with regard to FIG. 19A. However, comments relating to the core structure subassembly (340) are also applicable to the combination found in FIG. 19B. The cylindrical stub (338) extending from upper inner end plate (324) used to weld the inner upper and plate 324 to the upper outer endplate (328) as shown in FIG. 19, may also be seen. The compression member (322) has been included in core structure (340) but is hidden from view behind the various components. The subassembly (340) shown in FIG. 20 includes four cylinders of fiber (342) woven through the various slots or openings (344). The subassembly is made by passing a fiber (342) sequentially through a pair of adjacent slots (344) in one of the inner end plates (324, 326) shown in FIG. 19. The fiber (342) is then threaded past compression member (322), through the other inner end plate, and back through in adjacent slot in the end plate.

The fibers (342) shown in FIG. 20 are at a profound angle to the end plates (324, 326). This angle is chosen by selecting holes or slots (344) in the opposite inner end plates that produce the desired angle. Obviously, the angle of the fiber to the end plates changes with the radial distance from the center. In this depicted variation, the end plates (324, 326) have thirteen slots. In carrying out a pattern of weaving the fiber (342) through a first inner end plate and then through the opposing inner end plates, the pattern would entail skipping three openings (344) each time a fiber (342) is passed to an end plate. This variation entails weaving the fiber through the end plates (while skipping the noted number of slots) four times around the end plates (324, 326) to produce a single full woven cylindrical layer. There are four layers on that variation (shown in FIG. 20) of the core assembly. Choice of the number of slots or openings (344), typically an odd number, and choice of an appropriate number of “skipped” slots during weaving results in an appropriate layer.

The variation depicted in FIGS. 19A and 20, when designed for use in a cervical disc, includes upper and lower, Ti-6Al-4V alloy, inner end plates having a nominal diameter of about 0.475 inches (about 12 mm), an angle between adjacent slots of 28°, a slot width of about 0.030 inches (about 0.76 mm), a slot length of about 0.120 inches (about 3 mm) including inner radii, and a plate thickness of about 0.025 inches (about 0.635 mm). The core assembly in FIG. 20 was woven using four 20 inch sections of UHMW polyethylene fiber having a diameter of about 0.0185 to 0.022 inches, e.g., TELEFLEX—Force Fiber No. 2.

Similarly, the variation of our prosthetic disc as shown in FIG. 19B may comprise alloys such as Ti-6Al-4V alloy. That depicted variation, when used as a lumbar disc implant, may have a lateral dimension of about 34-38 mm. The heights of the lumbar implants, measured at the posterior edge of the disc, may be about 10-14 mm. When used in as lumbar disc implants, lordosis angles of 0° to about 15° would be typical.

The variations depicted in FIGS. 19A, 19B, and 20 have excellent resistance to the degradation of compressive stiffness whether the fibers are intentionally compromised or merely subjected to kinematic wear testing. We have tested the variation shown in FIGS. 19 and 20 for compressive stiffness both as produced and after significant testing: The compressive stiffness for samples fabricated with fiber layers compromised and tested in rotation ranged from 267 to 409N/mm. The compressive stiffness for the samples fabricated with fiber layers compromised and tested in compression shear with full extension ranged from 275 to 313N/mm. For the additional samples tested that had previously undergone kinematic wear testing, the compressive stiffness values were 407N/mm (10 million cycles, rotation), 448N/mm (10 million cycles, rotation), 390N/mm (20 million cycles, compression shear) and 416N/mm (20 million cycles, compression shear). In all cases, the values of compressive stiffness for the samples tested fell within the range for a native cervical disc.

A suitable fiber for the stress member comprises the UHMW polyethylene mentioned above. The fiber is used to attach the upper and lower inner endplates and, in some variations, to contain the compression member. As such, the fiber is subjected to tensile forces both during assembly and after implantation. An acceptance criterion of 100N takes into account anticipated assembly and clinical forces. Fibers exhibiting a tensile strength between 180 Nm and 210 Nm, e.g., between 183.8 Nm and 204.3 Nm, in a pull test to failure with an elongation rate of 30 cm per minute, are suitable for use in our device.

The variations shown in FIGS. 19A, 19B, and 20 desirably have a torsional resistance in the range of 0.10 Nm to about 0.55 Nm (outside of the neutral zone), often 0.12 Nm to 0.45 Nm, and 0.20 Nm to 0.45Nm.

### EXAMPLE

The biomechanical properties and characteristics of our prosthetic disc, in a cervical placement, were evaluated in a human cervical spine cadaver model. The purpose of the study was to assess the similarity of a total disc replacement, using our disc prosthesis, relative to a native human disc. The objectives of the study were to characterize the motion response of human cervical functional spinal units implanted with our artificial disc to moments applied in flexion-extension, lateral bending, and axial rotation and to assess the effect of the disc prosthesis on load-sharing through the anterior and posterior columns at the implanted and adjacent segments.

The prosthetic disc was studied relative to an intact human disc, in an age and disease-state appropriate cadaver spine, using a follower-load on a C3 to C7 cervical column. The study employed a follower-load model with 150 N preload through a 1.5 Nm bending moment. Baseline testing was completed on six samples in Flexion/Extension, Lateral Bending, and Axial Rotation for intact native disc versus our prosthetic disc.

Kinematic outcome measures included both the quality and quantity of motion. Quantity of motion was expressed as the range of motion (ROM) between +1.5 Nm and −1.5 Nm. The hysteresis curve or “loop” as shown in FIG. 15, illustrates characteristics of the quality of motion. Quality of motion is defined as abnormalities in the pattern of motion (as opposed to its magnitude). The quality of motion is further characterized by the following measurements: Neutral Zone (NZ), High Flexibility Ratios (HFR-F flexion, HFR-E extension), and Center of Rotation (COR).

For a complete follower-load background information, see, Patwardhan et al., “Load-Carrying Capacity of the Human Cervical Spine in Compression Is Increased Under a Follower Load,” Spine, vol. 25, 12:1548-1554. Briefly, a follower load is more representative of in vivo biomechanics by virtue of loading path. The load to the spine is applied through the centers of rotation of each vertebral body as opposed to a straight vertical load. This allows for tangential application of the load along the natural curve of the spine.

FIG. 21 depicts the test set-up for this method. A section of cadaver spine (400) is mounted firmly on the base (401). The spine section includes a number of vertebral bodies (402, 404, 406, 408, and 410). The natural discs between each of the vertebral bodies are left in place except for the prosthetic disc (412). The natural discs have been omitted from the Figure for simplicity. We placed eyelets in each vertebral body (402-410) to allow for passage of the loading cable (414) through the vertebral bodies. We have portrayed the cable (414) as exterior to the spine in the Figure, but it passes through the interior of the spine. The cable (414) is fixed to the uppermost vertebral body (402) with a fastener (416). The preload is applied at the bottom (418) of the cable around pulley (420).

The follower loading methodology loads the cervical spine curves in such manner that it simulates the native physiology. A 1.5 Nm moment is applied from the top of the assembly by virtue of a lever arm and a 150 N preload is applied through the cables, and an appropriately positioned pulley, from the bottom of the assembly.

The assessments were performed using six human cervical cadaver spines, age 51.5+5.5 years, including two (2) males and (4) females. None of the spines were osteopenic or osteoporotic. Kinematic assessments of biomechanical responses were collected including range of motion (ROM) or quantity of motion. The results, in part, are a hysteresis curve or “loop” demonstrating the quality of motion.

Six (6) cadaveric human spine specimens were tested both in the intact state and after implantation of our device at C5-C6. In each condition (intact and implanted) the specimens were subjected to the following loads: Flexion and extension moments (±1.5 Nm) with compressive preloads of 0 N and 150 N; lateral bending (±1.5 Nm) with compressive preloads of 0 N; axial rotation (±1.5 Nm) with compressive preloads of 0 N.

The prosthetic devices were implanted by a surgeon slightly posterior to the midline 0.9 mm±0.6 mm (Range: 0.3 to 1.9 mm posterior to the midline).

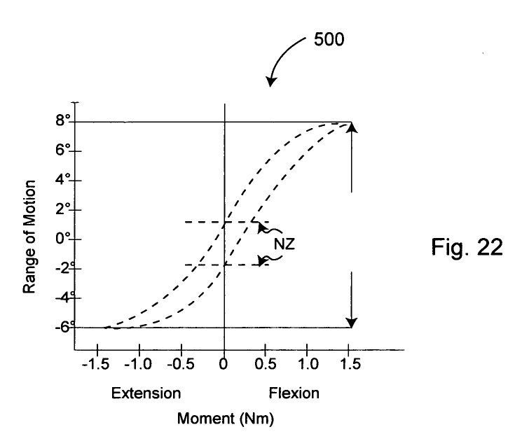

An apparatus allowing continuous cycling of the specimens between specified maximum moment endpoints in flexion, extension, lateral bending, and axial rotation was used. The motions of the vertebrae were measured using an optoelectronic motion measurement system, as well as bi-axial angle sensors. Load cells were placed under the specimen to measure the applied compressive preload and moments. Fluoroscopic imaging was used during flexion and extension to monitor vertebra and implant motion, and also used to measure segmental lordosis angles in the neutral posture under 150 nM compressive preloads. Spines were instrumented to monitor load sharing through the anterior and posterior columns. FIG. 22 provides a graph (500) showing the typical results from this testing.

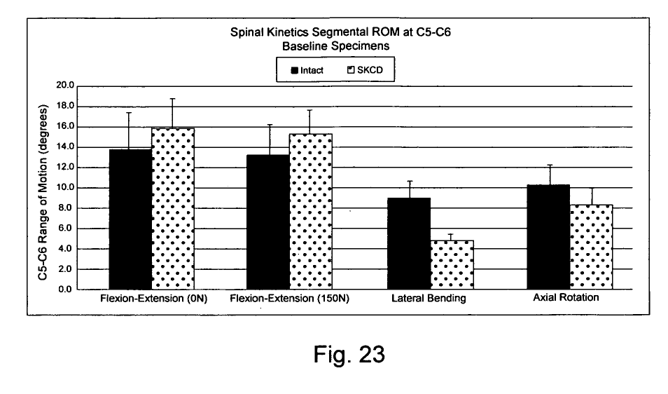

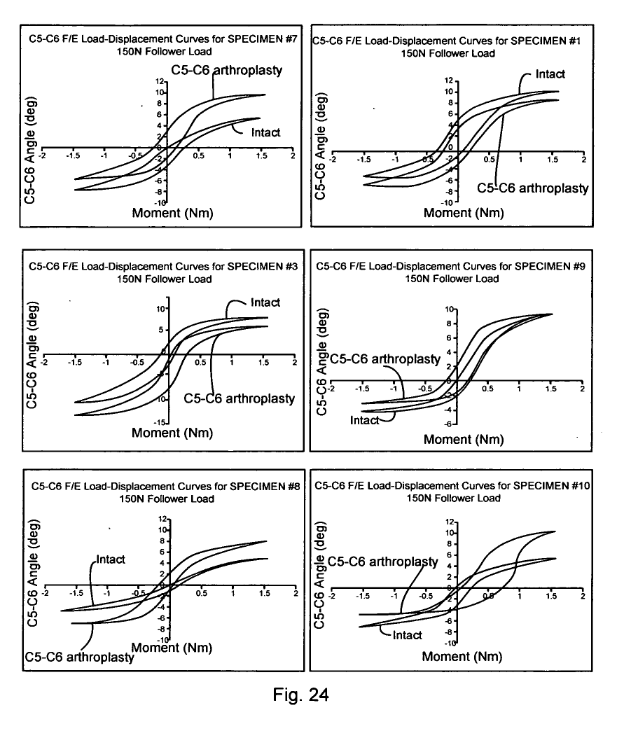

As seen in FIG. 23, with the exception of lateral bending, our prosthetic discs were within the physiologic range cited. Flexion/extension results were not statistically significant whereas lateral bending and axial rotation were statistically different from intact.

FIG. 24 shows the displacements, slopes, and neutral zone through the hysteresis loops for the intact disc versus implant disc in flexion and extension.

Our device improves the quality of motion by slightly increasing ROM, High Flexibility Ratio [HFR], Neutral Zone [NZ], and Hysteresis. One spine showed a limiting of extension and an expansion of flexion in the hysteresis loop for our device versus that for the intact specimen. This may be attributable to surgical preparation or potentially implanting a prosthesis that was too tall for this disc space.

There were no statistical differences between intact discs and our devices for HFR and NZ. The hysteresis data showed a difference between intact discs and our devices for the 150 N follower load treatment group. This suggests that on the whole our device is capable of absorbing more energy than the intact group studied.

In the 150N follower-load results, the native specimens were within the physiological F/E ROM with an average and standard deviation of 13.2°±3.1, and our devices typically resulted in physiological ROM with an average and standard deviation of 15.1°±2.5. The 0 N follower-load results for lateral bending show a ROM just under the reported in vivo active data. Two issues are at play in this lateral bending ROM result. First, the test was conducted at 0 N due to limitations of the loading technique using bilateral cables. The in vivo load is in the 70 N-to-150 N range, which may have an effect on ROM. Second, this reduction in ROM is reflective of uncinate process phenomenon expressed in total disc replacement. Upon native disc removal and implantation of our prosthesis, the center of rotation (COR) moves towards the geometric center of the implant instead of remaining near the superior vertebral body's lower endplate. This reduces the ROM, because the lateral motion trajectory is altered from swinging motion to a tilting motion. This alteration of motion allows the uncinate processes to come in contact thereby limiting motion. Ongoing research suggests that resection of the uncinate processes allows restoration of full ROM. For axial rotation, these ROM differences are small, and there should be no clinical consequence because our disc is within or very close to the in vivo active range cited.

Based upon analysis of segmental ROM, HFR, NZ, Hysteresis, and disc pressures, our prosthetic disc has biomechanical performance similar to that of a native human disc. Our disc restored the quantity and quality of motion to physiologic norms in flexion/extension, and the intradiscal pressure was not affected at the adjacent levels. A notable difference was found in lateral bending ROM, which was likely due to the caudal migration of COR not uncommon to total disc replacement. Also, the prosthetic disc in one spine had an expanded flexion loop relative to extension, which may be due to surgical technique or the height of the implant. Another difference found was the increase in hysteresis of the spines incorporating our disc. This demonstrates our disc's ability to absorb slightly more energy than the native disc. In this cadaver model the data show that our disc provides similar biomechanics to the lower cervical spine as compared to the intact spine.

### EXAMPLE

Another cadaver spine segment was later installed on the test rig. In this instance, the segment included L-1 to the Sacrum. The segmental and total ROM in flexion-extension were measured at various follower loads. The values were measured both with the native discs all intact and then with the implant inserted at L4-L5. The implant was of the design shown in FIG. 19B, having a width of 34 mm. The following results were obtained:

### Total Flexion-Extension ROM

### Measurements in °

### 0 N Follower Load

TOTAL FLEXION-EXTENSION ROM
(Measurements in °)

| | | | | | |
|---|---|---|---|---|---|
| L1-S1 | L1-L2 | L2-L3 | L3-L4 | L4-L5 | L5-S1 |

0 N Follower Load

| | | | | | | |
|---|---|---|---|---|---|---|
| Intact | 46.2 | 8.1 | 7.1 | 7.0 | 12.4 | 11.3 |
| Implant | 49.0 | 9.7 | 8.8 | 9.2 | 9.6 | 11.3 |
| at L4-L5 | | | | | | |

400 N Follower Load

| | | | | | | |
|---|---|---|---|---|---|---|
| Intact | 43.8 | 7.7 | 7.4 | 7.6 | 11.7 | 9.2 |
| Implant | 41.7 | 7.7 | 8.2 | 8.4 | 8.2 | 8.8 |
| at L4-L5 | | | | | | |

800 N Follower Load

| | | | | | | |
|---|---|---|---|---|---|---|
| Intact | 41.4 | 7.6 | 7.5 | 7.7 | 10.8 | 7.8 |
| Implant | 41.5 | 8.3 | 8.5 | 8.4 | 8.3 | 8.0 |
| at L4-L5 | | | | | | |

In this cadaver model demonstration, the data show that our disc provides similar biomechanics to the lumbar spine as compared to the intact lumbar spine.

We claim as our invention:

1. A prosthetic intervertebral disc comprising:
a first end structure attachable to a first vertebrae,
a second end structure attachable to a second vertebrae,
a core structure comprising at least a portion in compression with relation to the first end structure and the second end structure, said core member comprising at least a portion in tension with relation both to the first end structure and to the second end structure, having a bulk compressibility of 737 Nmm+/−885 Nmm, and positioned with respect to and interacting with the first end structure and with the second end structure such that, when measured with 150 Nm axial preloading, provides:
a nonlinear torsional response to relational movement between the first end structure and with the second end structure when a torsional moment is applied to at least one of the first end structure and the second end structure of the form in FIG. 5, and,
a nonlinear side-to-side bending response to relational movement between the first end structure and the second end structure when a side-to-side bending moment is applied to at least one of the first end structure and the second end structure of the form in FIG. 5, and,
a nonlinear flexion-extension to relational movement between the first end structure and the second end structure when a flexion-extension moment is applied to at least one of the first end structure and the second end structure of the form in FIG. 5, and,
and wherein each of the first end structure and the second end structure defines an IAR and wherein the IAR's of each of the first end structure and the second end structure, when a moment is applied to said end structure, is determined by the compression and tension of the core structure

2. The prosthetic intervertebral disc of claim 1 wherein the first end structure and the second end structure are substantially inflexible.

3. The prosthetic intervertebral disc of claim 1 wherein at least one of the first end structure and the second end structure are directly attachable respectively to a first vertebrae and to a second vertebrae.

4. The prosthetic intervertebral disc of claim 1 wherein the first end structure and the second end structure are indirectly attachable respectively to a first vertebrae and to a second vertebrae.

5. The prosthetic intervertebral disc of claim 1 wherein the portion of the core structure in compression comprises at least one polymeric elastic member having a bulk compressibility of 737 N/mm+/−885 N/mm extending between the first end structure and the second end structure.

6. The prosthetic intervertebral disc of claim 1 wherein the portion of the core structure in tension with relation both to the first end structure and to the second end structure comprises multiple polymeric fibers extending between the first end structure and the second end structure and having a tensile strength between 180 and 210 Nm.

7. The prosthetic intervertebral disc of claim 1 wherein the portion of the core structure in tension with relation both to the first end structure and to the second end structure comprises multiple polymeric fibers extending between the first end structure and the second end structure and configured to provide torsional resistance between the first end structure and the second end structure with a neutral zone and having torsional resistance of about 0.10 Nm to about 0.55 Nm outside of the neutral zone.

8. The prosthetic intervertebral disc of claim 1 wherein the disc neutral zone is +4° to −2.5° in flexion-extension.

9. The prosthetic intervertebral disc of claim 1 wherein the disc ultimate range of motion (ROM) limit is 10° to 13° in flexion-extension.

10. The prosthetic intervertebral disc of claim 1 wherein the disc ultimate range of motion (ROM) limit is 8° to 10° in lateral movement.

11. The prosthetic intervertebral disc of claim 1 wherein the disc ultimate range of motion (ROM) limit is 8° to 10° in axial rotation.

12. The prosthetic intervertebral disc of claim 1 wherein the at least a portion of the compressible core member in compression comprises a barrel-shaped polymeric member.

13. The prosthetic intervertebral disc of claim 12 wherein the compressible core member in compression comprises a barrel-shaped polymeric member formed by compression molding and heat-treating.
